(19) United States
(12) Patent Application Publication
(10) Pub. No.: US 2019/0282175 A1
(43) Pub. Date: Sep. 19, 2019

(54) SYSTEMS AND METHODS FOR VALIDATING SENSOR DATA

(71) Applicant: IXICO Technologies Limited, London (GB)

(72) Inventor: Derek Lionel Glendon Hill, London (GB)

(73) Assignee: IXICO Technologies Limited, London (GB)

(21) Appl. No.: 16/318,838

(22) PCT Filed: Jul. 24, 2017
(86) PCT No.: PCT/GB2017/052152

§ 371 Date: Jan. 18, 2019

(30) Foreign Application Priority Data
Jul. 22, 2016 (GB) 1612721.9

(51) Int. Cl. A61B 5/00; G06F 3/01; G06F 17/18; G06F 21/32; B25J 9/16

(52) U.S. Cl. CPC A61B 5/7221; G06F 3/015; B25J 9/1694; G06F 17/18; G06F 21/32; G06F 3/017

(57) ABSTRACT

A system for validating biosensor data, the system comprising: i) means for collecting biosensor data from a subject; ii) means for reproducing subject behaviour and/or physiological activity of the subject and collecting data corresponding with such reproduced behaviour and/or physiological activity; and iii) means for detecting any deviation over time in biosensor data collected from the reproduced activity in (ii) in order to identify and/or correct sensor or software introduced deviations in biosensor data collected from the patient.

### FIELD

The present invention relates to systems and methods for validating sensor data, particularly, but not exclusively, biosensor data.

### BACKGROUND

In digital healthcare sensor applications there is a distinct difference between three types of data: i) consumer grade; ii) medical grade; and iii) research grade.

Consumer grade biosensor data, for example from smart phone apps, home-based sensors and wearable devices, can be collected in large volumes from widely adopted technologies. These devices have, however, often undergone very little validation to demonstrate how meaningful the measurements are, and to characterise their accuracy. Furthermore, it is not clear how stable the measurements are over time, as the software environment of the sensors is often continuously evolving. This can be because the software on the sensor itself (eg: on a wearable), or software in the ecosystems (smartphone app, smart phones OS, cloud servers etc.) is upgraded automatically. It is possible that one reason for this change in software environment is that the sensors or surrounding ecosystem may be “self learning” and alter their performance as they gather more data.

Medical grade biosensor data is collected as part of formal healthcare service provision. This data is normally collected by medical devices, with well characterised performance, and formally validated software that must be updated in a very controlled way. But this data is often limited and sparse compared with what can be obtained from consumer devices, and the devices have less functionality, are less user-friendly, and generally have shorter battery life.

Research grade biosensor data is collected in research studies e.g.: evaluating new drugs or devices or studying the natural history of diseases. The biosensors used in these studies may not be medical devices, but need to have well characterized performance, for example in order to power the study. Similarly the use of the device needs to be controlled to ensure comparable data from different subjects and over time. For all three types of biosensor, obtaining actionable information to inform decision making in clinical trials and patient management requires validation of the measurement for the specific intended purpose, and the ability to calibrate between different devices, e.g. following upgrades of software or hardware. Furthermore, it is desirable to replace one sensor with another one during the course of a clinical trial or the management of a patient, and to ensure the data from the original and replacement sensors is comparable.

The present invention seeks to solve the aforementioned problems by providing systems and methods for validating sensor data such as data obtained from devices including wearable devices and smart phones, for example.

### SUMMARY

An aspect of the invention provides a system for validating biosensor data, the system comprising: i) means for collecting biosensor data from a subject; ii) means for reproducing subject behaviour and/or physiological activity of the subject and collecting data corresponding with such reproduced behaviour and/or physiological activity; and iii) means for detecting any deviation over time in biosensor data collected from the reproduced activity in (ii) in order to identify and/or correct sensor or software introduced deviations in biosensor data collected from the subject.

The ability to use data to support research and clinical practice, as this data is much richer and more available than traditionally available medical grade and research grade data, is highly advantageous in improving the quality of research, regulatory submissions and subject care. The system defined by this aspect of the invention enables professionals to use consumer grade data with confidence due to the ability to verify the accuracy of collected data regardless of the type or brand of biosensor used to collect data.

The system may further comprise means to apply a correction factor to any deviation identified between biosensor data collected from the subject and data corresponding to reproduced behaviour.

Another aspect of the invention provides a method of validating sensing data, the method comprising: a) obtaining behavioural and/or physiological data from a test subject through a first sensor; b) using said behavioural and/or physiological data to program a device to replicate behaviour and/or physiological attributes exhibited by the test subject; c) applying a second sensor to the device; d) using the device to replicate the test subject's behaviour and/or physiological attributes; e) obtaining behavioural and/or physiological data from the second sensor; and f) identifying any variation between the data obtained by the first sensor and the data obtained by the second sensor.

Another aspect of the invention provides a method of simultaneously validating sensing data from multiple sensors, the method comprising a) obtaining behavioural and/or physiological data from a test subject through a first sensor; b) using said behavioural and/or physiological data to program a device to replicate behaviour and/or physiological attributes exhibited by the test subject; c) applying multiple additional sensors to the device; d) using the device to replicate the test subject's behaviour and/or physiological attributes; e) obtaining behavioural and/or physiological data from the additional sensors; and f) identifying any variation between the data obtained by the first sensor and the data obtained by the additional sensors.

Use of the terms patient and subject are used interchangeably throughout and may relate to clinical patients, athletes or placebo subject, for example.

### FIGURES

Embodiments of the inventions will now be described by way of reference to the following figures:

FIG. 1 illustrates problems associated with uncontrolled data collected from consumer sensors;

FIG. 2 illustrates a first method of validating sensor data;

FIG. 3 illustrates a second method of validating sensor data.

### DESCRIPTION

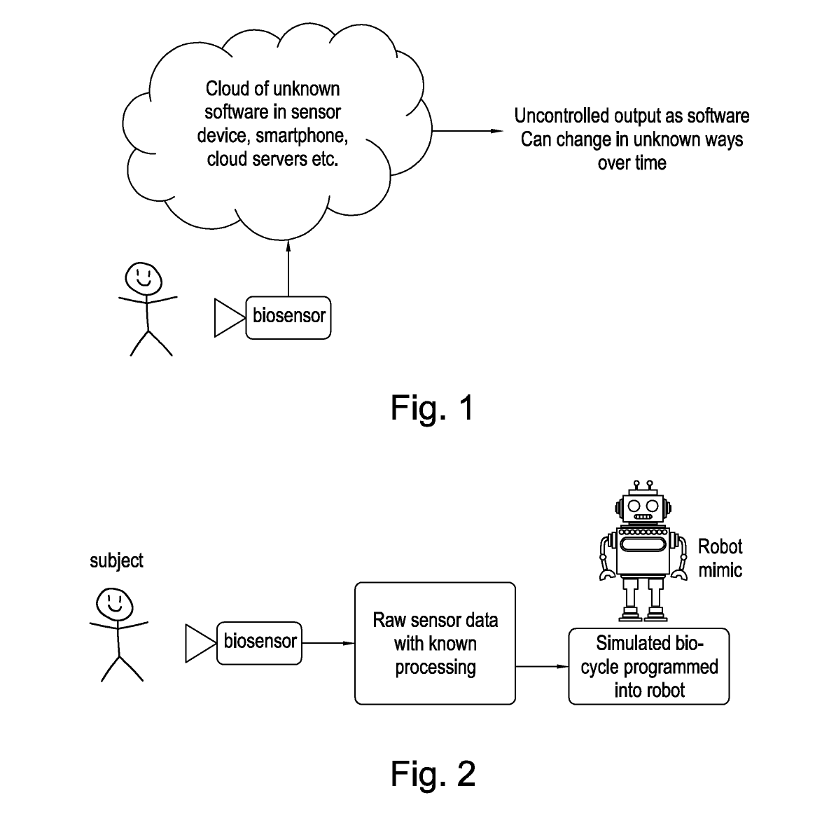

The technical problem is indicated schematically in FIG. 1. The biosensor (which might be a wearable, wall mounted or static device, or built into a smart-phone) makes measurements that then pass through a number of stages of processing on the sensor device, on any smart phone involved in the chain, and in any file server the data is transferred to. In FIG. 1, those various software processes and data transfers are represented as a “software cloud”. The output is uncontrolled because what happens in the cloud can change in an unknown way at unknown times.

Key technical challenges need to be overcome to achieve the goal of using biosensors for regulated applications in clinical research and subject management, for example in clinical trials of new drugs, and as digital healthcare companion products to drugs. This requires addressing the issues of system validation, and quality assurance of data that is obtained out of this software cloud, in particular so any change over time in the outputs that is caused by the hardware software changes, device failure or improper use can be detected and corrected for.

Embodiments of the present invention describe a solution to the aforementioned technical challenges.

Rather than the validation of the biosensor being done before the device is used, with a carefully controlled process for upgrading software if any changes are required, the validation becomes a continuous process while data is being collected. This requires a standardised input of sensor data from a laboratory environment into the same software cloud that is handling the subject data.

This standardised input involves the use of a robot mimic. For the purposes of this application, “Robot Mimic” means a computer controlled system that, when connected to or measured by the sensor, generates sensor data that is highly correlated with that generated by the clinical trial subject/patient when connected to or measured by the same sensor. FIG. 2 illustrates one example of how the robot mimic may be set up using actual biosensor data. Raw data from the biosensor is used to set up a simulated bio-cycle in the robot mimic.

The simulated bio-cycle determined using the approach illustrated in FIG. 2 can be run through the robot mimic as often as required and the robot mimic has sufficient reproducibility to ensure that it performs with a reproducibly that is much better than the measurements accuracy required by the application.

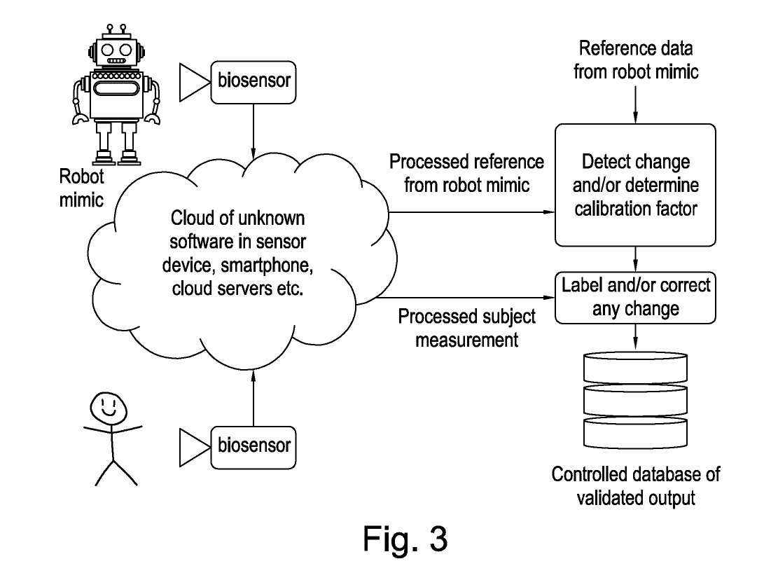

The overall validation of the biosensor measurements requires that measurements from the robot mimic are collected periodically or continuously while the clinical trial subject/patient data is being collected. During this validation process, the same type of biosensor, or biosensor(s), are connected to subject and robot mimic, and the same software cloud is used for the analysis of all data collected. Biosensors used in embodiments of the invention can be any type of biosensor, including consumer grade biosensors “out of the box” as illustrated in FIG. 3.

Performance of the system can be improved by using multiple robot mimics rather than just one in parallel with the data collection from the subjects.

It is also possible for multiple sensors (eg: different brands of activity watch) to be attached simultaneously to the same robotic mimic to enable the clinical trial subjects/patients to use different sensors in the same study, with standardisation across those sensors provided by the system.

Biosensors that could be validated by embodiments of the invention include, but are not limited to. temperature, blood pressure, speech, activity, and social connectivity (proximity to another sensor.

The invention can also optionally acquire baseline reference data from the subjects or clinical trial subjects under investigation, for example during a set-up phase in a clinical environment while the subject or clinical trial subjects is being observed by a medical professional or a video recording system performing activities relevant to that sensor.

An embodiment of the invention involves a sensor that measures sleep from patients with a particular pathology (eg: Parkinson's disease), using three axis accelerometers included in a wrist-worn device. This involves the following steps:

1. Collecting representative sensor data from subjects who are undergoing polysomnography (PSG) while wearing sensor A. This data can be used to train a algorithm such that sleep measures derived from sensor A can be made comparable to measures of sleep from PSG.

2. Programming the robotic mimic (a robot arm) with sensor data from the step 1, so that the robot mimic can replicate arm movements during sleep that were equivalent to those movements conducted by the subjects undergoing PSG.

Step 3. Calibration of a different 3-axes accelerometer based biosensor (eg: one that has a longer battery life), sensor B, using the robot mimic programmed in step 2. This calibration would enable sleep measures derived from sensor B to be made comparable to measures of sleep from PSG, just had been done for Sensor A. And this would have been achieved without Sensor B ever having been worn by subjects undergoing PSG.

A second embodiment of the invention involves a biosensor that measures skin temperature and activity and which sends this information via low energy blue-tooth to a smart phone, from where it is sent via either the mobile phone air interface or WiFi to a cloud server, and then via an application programming interface (API) provided by the sensor supplier to a controlled database to be used for research or clinical purposes. During these various data transfers the biosensor data is compressed so that all that is delivered to the controlled database is information on number of steps, amount of deep and light sleep, and skin temperature. It is the data in the controlled database that needs to be validated for the purpose for which it is being used. This involves four stages:

Step 1—Collecting a representative test sensor dataset: biosensors which collect raw data (eg: actual accelerometer readings rather than steps) are attached to one or several individuals who then undertook the expected range of activities that would be undertaken by the sensor user (walking, running, travelling on public transport and in a car, sleeping, resting etc). The range of movements expected in the population to be studied would be included in this test sensor data, for example different gaits, width of stride etc. Raw data from the sensors will be collected over a sufficient period of time (approx. forty eight hours per subject) to be representative of a subject's behaviour and activities.

Step 2—Programming a robotic mimic as a sensor phantom: the representative sensor data from step 1 (above) is used to programme the controller of a robot with the appropriate number of degrees of freedom, precise optically encoded motors and with a precisely controlled variable temperature patch. The robotic mimic is programmed with one or more simulated bio-cycles derived from the data collected in step 1. The consumer biosensor(s) are then attached to this robot mimic, so that it collects data that mimics data from sensors attached to an actual clinical trial subject/patient such that the raw data coming out of the biosensor would be highly correlated with the raw data collected in step 1. This robotic mimic is sufficiently precise and reproducible that it can reproduce the same pattern of movement and temperature change each time the simulated bio-cycle is repeated, with errors that are smaller than the acceptable measurement error required for the application. This robotic mimic can also optionally be used to provide absolute calibration of the biosensor measurement.

Step 3—Collect baseline performance data from the sensor: the biosensor(s) are attached to the robot mimic, operating in a normal mode in which data passes through the software cloud (i.e.: data collected from the biosensor(s) are sent by low energy to a smart phone which sends by WiFi to the cloud server from which it is extracted to the controlled database using an API) while the robot mimic performs the simulated bio-cycle. Baseline data is collected from the output of the software cloud for the sensors, along with test, re-test and inter-device data by repeating the simulated bio-cycle multiple times with multiple versions of the device.

Step 4—Real-time quality assurance and validation: while biosensor data is being collected from subjects involved in a research study or from subjects in clinical practice, biosensor data from a simulated bio-cycle is also periodically collected (e.g.: daily) from the robot mimic, with the data from both the subjects and robot mimic passing through the same software cloud. The change in measurements from each bio-cycle of the robot mimic is compared to the baseline performance data to detect any change in measurements that is more than a pre-determined difference (eg: 1.5 standard deviations) away from the measured test, re-test performance. If there is any upgrade in the software cloud (e.g.: the software running on the device, or on the smart-phone app, or on the cloud servers) which impacts the measurements, this would be detected from that bio-cycle comparison. The output of the comparison is linked to the subject biosensor data, and can either be used to quantify the magnitude of any change in the sensor output from the biocycle for use in statistical analysis, or can be used to apply a calibration factor to the subject data so that changes in output due to variability in the software cloud are corrected for.

In a further embodiment of the invention, the method allows for authentication of the subject or research subject being studied. According to this embodiment, representative test sensor data is collected from each individual subject or research subject being investigated under standardized conditions (eg: in a clinic)—“subject reference data”. This subject reference data may replace or augment the test data described in step 1 above. The system can then compare data collected regulatory or continuously from this subject over time, and compare to the subject reference data. If changes over time are large that indicates that sensor may no longer be monitoring the intended subject and therefore, and if the ongoing measurements are sufficiently consistent with the original subject reference data, that indicates that the same subject is being studied throughout the period. The sensor data might, for example, be the subject's speech, or gait information derived form an actigraphy sensor.

1: A system for validating biosensor data, the system comprising:
i) means for collecting biosensor data from a subject;
ii) means for reproducing subject behaviour and/or physiological activity of the subject and collecting data corresponding with such reproduced behaviour and/or physiological activity; and
iii) means for detecting any deviation over time in biosensor data collected from the reproduced activity in (ii) in order to identify and/or correct sensor or software introduced deviations in biosensor data collected from the subject.

2: The system according to claim 1, wherein the means for collecting biosensor data from a subject comprises a temperature sensor, motion sensor, heart rate sensor, pressure sensor, microphone or position sensor.

3: The system according to claim 2, wherein the means for collecting biosensor data comprises one or more sensors forming part of a smart phone, watch, garment or other subject mounted sensor.

4: The system according to claim 1, wherein the means for reproducing subject behaviour and/or physiological activity of the subject comprise a programmable device or robot.

5: The system according to claim 4, wherein the means for collecting data corresponding with reproduced behaviour and/or physiological activity comprise one or more sensors attached to the robot or capable of remotely monitoring the robot.

6: The system according to claim 5, wherein the subject behaviour and/or physiological activity data is temperature and the means for reproducing said subject behaviour and/or physiological activity is a programmable temperature device.

7: The system according to claim 5, wherein the subject behaviour and/or physiological activity is speech and the means for reproducing said subject behaviour and/or physiological activity is a programmable audio device.

8: The system according to claim 5, wherein the subject behaviour and/or physiological activity is positioning relative to one or more reference sensors and the means for reproducing said subject behaviour and/or physiological activity is a robot.

9: The system according to claim 5, wherein the means for reproducing subject behaviour and/or physiological activity is a simulated programmable device.

10: The system according to claim 1 further comprising means to apply a correction factor to any deviation identified between biosensor data collected from the reproduced behaviour over time.

11: The system according to claim 1, wherein data collected from the subject and/or means for reproducing subject behaviour and/or physiological data is raw data.

12: A method of validating sensing data, the method comprising:
a) obtaining behavioural and/or physiological data from a test subject through a first sensor;
b) using said behavioural and/or physiological data to program a device to replicate behaviour and/or physiological attributes exhibited by the test subject;
c) applying a second sensor to the device;
d) using the device to replicate the test subject's behaviour and/or physiological attributes;
e) obtaining behavioural and/or physiological data from the second sensor regularly over time; and
f) identifying any variation between the data obtained by the first sensor and the data obtained by the multiple measurements made over time with the second sensor.

13: The method according to claim 12, further comprising the step of g) applying a correction factor to data obtained from the second sensor in the event that any variation between data obtained from the first sensor and data obtained from the second sensor exceeds a predetermined threshold.

14: The method according to claim 12, wherein the step of using the device to replicate the subject's behaviour and/or physiological activity is repeated multiple times to improve accuracy of replication through machine learning.

15: The method according to claim 13 further comprising the step of h) using said correction factor to calibrate the second sensor.

16: The method according to claim 12 where the test subject is the subject themselves, having data collected in standardized conditions prior to being monitored in a real world setting such as their home.

17: The method according to claim 16 where change over time in the sensor measurements authenticates the subject by detecting whether the sensor is still being worn by the same subject.

18: A method of simultaneously validating sensing data from multiple sensors, the method comprising:
a) obtaining behavioural and/or physiological data from a test subject through a first sensor;
b) using said behavioural and/or physiological data to program a device to replicate behaviour and/or physiological attributes exhibited by the test subject;
c) applying multiple additional sensors to the device;
d) using the device to replicate the test subject's behaviour and/or physiological attributes;
e) obtaining behavioural and/or physiological data from the additional sensors; and
f) identifying any variation between the data obtained by the first sensor and the data obtained by the additional sensors.
